(19) United States
(12) Patent Application Publication
(10) Pub. No.: US 2018/0368664 A1
(43) Pub. Date: Dec. 27, 2018

(54) ROBOTIC MATERIALS AND DEVICES

(71) Applicant: DynamicSurgical Inc., Saint Louis, MO (US)

(72) Inventors: Danish NAGDA, Town and Country, MO (US); Jeffrey GAMBLE, St. Louis, MO (US); Ilker TUNAY, St. Louis, MO (US)

(21) Appl. No.: 16/018,745

(22) Filed: Jun. 26, 2018

Related U.S. Application Data
(60) Provisional application No. 62/525,107, filed on Jun. 26, 2017.
(60) Provisional application No. 62/545,255, filed on Aug. 14, 2017.
(60) Provisional application No. 62/652,418, filed on Apr. 4, 2018.
(60) Provisional application No. 62/672,803, filed on May 17, 2018.

(51) Int. Cl. A61B 1/00; A61M 29/02; A61B 18/18; A61B 1/005

(52) U.S. Cl. CPC A61B 1/00133; A61M 29/02; A61B 18/1815; A61B 1/00135; A61B 2018/00577; A61B 1/00158; A61B 1/00156; A61B 1/0058; A61B 1/0051; A61B 1/00078

(57) ABSTRACT

Embodiments of the present disclosure generally relate to variable stiffness materials and devices, and methods of use thereof. In one embodiment, a variable stiffness robotic material is disclosed, which in one example, is useful for forming a robotic material based sleeve for endoscopes. In another embodiment, a single tool variable stiffness endoscope and working channel is disclosed, which is useful for performing multi-site thermoblation in a physician's office. In yet another embodiment, a micro-wave based tissue ablation or volume reduction tool and procedure are provided for treating sleep apnea.

### CROSS-REFERENCE TO RELATED APPLICATIONS

This application claims benefit of U.S. provisional patent application Ser. No. 62/525,107, filed Jun. 26, 2017, U.S. provisional patent application Ser. No. 62/545,255, filed Aug. 14, 2017, U.S. provisional patent application Ser. No. 62/652,418, filed Apr. 4, 2018, and U.S. provisional patent application Ser. No. 62/672,803, filed May 17, 2018, each of which is herein incorporated by reference in its entirety.

### BACKGROUND

### Field

Embodiments of the present disclosure generally relate to robotic materials and devices, and methods of use thereof, including but not limited to a robotic material enabled medical device with embedded sensing, computation, and actuation.

### Description of the Related Art

Robotic materials are composite materials that are fully-programmable using the integration of sensing, actuation and computation to change properties, such as shape, volume, stiffness or physical appearance, of the underlying material(s). Variable stiffness materials are one type of robotic materials having a stiffness that can be changed from a flexible to a more rigid state. Such variable stiffness materials are useful for a variety of applications, including, but not limited to, vibration dampening in aeronautics and automotive applications, deployable interfaces in electronics and construction applications, resistive or supportive wearables in training and rehabilitation applications, as well as for medical devices and procedures.

Current endoscopic procedures, specifically laryngoscopies, often involve doctors using flexible scopes to visualize difficult to reach areas while maintaining patient comfort. However, flexible scopes lack the rigidity that is needed for controlled manipulation or puncture of tissue in minimally invasive procedures. It is desirable to provide a device which can switch from a flexible state to a much stiffer one at an operator's discretion. Additionally, it is desirable to design a device able to be incorporated with existing endoscopes, the majority of which do not have the working channels necessary for inserting minimally invasive surgical tools, such as ablation catheters, thus preventing the need for significant investment into new pieces of equipment.

Therefore, there is a need for improved robotic materials and devices that can be used for various applications, such as medical procedures in which the physician can reversibly modify, as needed, the stiffness of the materials comprising the surgical tools across a spectrum of flexible to rigid states.

### SUMMARY

Embodiments of the present disclosure generally relate to variable stiffness materials and devices, and methods of use thereof. In one embodiment, a variable stiffness robotic material is disclosed which, in one example, is useful for forming a robotic material based sleeve for endoscopes. In another embodiment, a single tool variable stiffness endoscopic overtube and working channel is disclosed, which is useful for performing multi-site thermoblation in a physician's office when coupled with a flexible endoscope. In yet another embodiment, a micro-wave based tissue ablation or volume reduction tool and procedure are provided for treating sleep apnea.

In one embodiment, a variable stiffness robotic material cell is disclosed. The variable stiffness robotic material cell includes a first compression sheet and a second compression sheet and a plurality of thin sheets of material arranged in a stack between the first compression sheet and the second compression sheet, each pair of adjacent thin sheets of the plurality of thin sheets having friction therebetween.

In another embodiment, a variable stiffness endoscope overtube is disclosed. The endoscope overtube includes one or more channels and an actuation layer disposed around at least one of the one or more channels. The actuation layer includes a plurality of variable stiffness robotic material cells. Each variable stiffness robotic material cell includes a first compression sheet and a second compression sheet, and a plurality of thin sheets of material arranged in a stack between the first compression sheet and the second compression sheet, each pair of adjacent thin sheets of the plurality of thin sheets having friction therebetween.

In yet another embodiment, a variable stiffness endoscope overtube is disclosed. The endoscope overtube includes one or more channels and consists of rigid joints, joining flexible segments, each of which is constructed by assembling rigid rings over the length of an inner tube, and lines that are threaded through the rings traversing the entire segment and terminating at joints. The joints may be articulated linearly or rotationally or they may be fixed. At each end of the overube, the lines are attached to rigid terminals. The stiffness of each segment is increased greatly by holding the lines so as to prevent their sliding through the rings in the axial direction, and reduced by the same amount by releasing the lines. The lines are kept in tension at all times by either spring loading one or both ends of a line inside a joint or by a slack removal mechanism.

In yet another embodiment, a method is disclosed. The method includes sliding the variable stiffness endoscope overtube over the working length of the flexible scope. The flexible scope covered with the variable stiffness endoscope overtube are inserted through the nasal passage of a patient to a first treatment site, the variable stiffness endoscope tube either 1) having an actuation layer having a plurality of variable stiffness robotic material cells, actuating the actuation layer of the variable stiffness endoscope overtube to increase the rigidity of the variable stiffness endoscope overtube or 2) having rigid ring segments connected by flexible segments, actuating to hold the lines passing through the rings from moving axially to increase the rigidity of the variable stiffness endoscope overtube, and performing thermoblation of a first tissue at the first treatment site using the rigid variable stiffness endoscope overtube in conjunction with an ablation catheter that has been inserted through one of the variable stiffness endoscope overtube's channels.

In yet another embodiment, a method is disclosed. The method includes sliding the variable stiffness endoscope overtube over the working length of a flexible endoscope, inserting the endoscope and variable stiffness endoscope overtube combination through the nasal passage of a patient to a first treatment site, the treatment site being selected from the group consisting of the nose, pallet, tongue and epiglottis, and delivering microwaves to the first treatment site to ablate a first submucosal tissue at the first treatment site once sufficient stiffness has been achieved for application of submucosal ablation.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 depicts a variable stiffness robotic material cell according to embodiments disclosed herein.

FIGS. 2A-2B depict electropermanent magnets according to embodiments disclosed herein.

FIGS. 3A-3C depict cross-sectional views of a plurality of jamming layers according to embodiments disclosed herein.

FIGS. 4A-4D depict an endoscope overtube according to embodiments disclosed herein.

FIGS. 5A-5E depict an endoscope overtube assembly according to embodiments disclosed herein.

FIGS. 6A-6N depict an endoscope overtube assembly according to embodiments disclosed herein.

FIG. 7 is a flow chart of a method according to embodiments disclosed herein.

FIG. 8 is a flow chart of a method according to embodiment disclosed herein.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments of the present disclosure generally relate to variable stiffness materials and devices, and methods of use thereof. In one embodiment, a variable stiffness robotic material is disclosed, which in one example, is useful for forming a robotic material based sleeve for endoscopes. In another embodiment, a single tool variable stiffness endoscope overtube and working channel is disclosed, which is useful for performing multi-site thermoblation in a physician's office. In yet another embodiment, a micro-wave based tissue ablation or volume reduction tool and procedure are provided for treating sleep apnea.

FIG. 1 depicts a robotic material cell 100 according to embodiments disclosed herein. In operation, a property of the robotic material cell 100, such as shape, volume, stiffness or physical appearance, is changed using actuation, sensing, and computation components that are embedded within the robotic material cell 100. Robotic material cells can be arranged next to one another such that they form an array of cells in various conformations. Individual cells within an array can be coupled such that nearby by cells exchange information about their respective states and can adjust their states accordingly. In one example, the rigidity of the robotic material cell 100 is changeable on demand, giving the robotic material cell 100 variable stiffness. The variable stiffness robotic material cell 100 is useful for a variety of applications. The variable stiffness robotic material cell 100 generally includes three components, an actuator mechanism 102, at least one sensor 104 and a computation component 106. The robotic material cell 100 may further include communication components. In operation, the computation component 106 activates the actuator mechanism 102. One or more sensors 104 measure output or other environmental stimuli or data that may be fed back into the computation component 106 for the continued use of the variable stiffness robotic material cell 100.

In the example of the variable stiffness robotic material cell 100, the actuator mechanism 102 is a variable stiffness actuator mechanism. The variable stiffness actuator mechanism 102 is generally any suitable mechanism which causes a change in the rigidity of the variable stiffness robotic material cell 100. Layer jamming, for example, provides scalable rigidity based on surface interactions between sheets of material and the normal direction (perpendicular) pressure applied to two or more sheets. As the individual sheets slide past one another during application of a bending force thereto while experiencing low pressure or force in the direction normal thereto tending to press the sheets together, the variable stiffness robotic material cell 100 is pliable. As the normal to the surface direction pressure of force is increased, the sheets lose the ability to slide past one another and undergo a phase transformation experienced as a stiffening or retention of the shape of the variable stiffness robotic material cell 100 even under the application of the normal to the surface direction pressure or force. Pressure is generally applied, directly or indirectly, to the sheets of material using magnetic systems, pneumatic systems, hydraulic systems, mechanical systems, electrostatic systems and formable materials systems. Similarly, particle jamming provides scalable rigidity based on the density of particles. Rheological materials that change physical state very quickly in response to a stimulus also provide scalable rigidity and are useful as the variable stiffness actuator mechanism. Even further, the present disclosure contemplates using other mechanical or material-based actuator mechanisms, such as electroactive polymers (EAPs) and shape memory alloys (SMAs), to cause a change in the rigidity of the variable stiffness robotic material cell 100.

The computation component 106 generally includes an open-loop or closed-loop computer input signal such that the variable stiffness robotic material cell 100 is a programmable system. In operation, the computation component 106 provides instructions for actuating the variable stiffness actuator mechanism 102. The computation component 106 generally includes a power source, one or more capacitors, and one or more controls. The power source is generally any suitable power source, including a battery or wall, i.e., facility, power source. The one or more capacitors can be distributed capacitors for each cell. The one or more controls can be any suitable controls which provide the input signal for actuation of the variable stiffness robotic material cell 100. Suitable controls include, but are not limited to, switches, potentiometers, and pulse-based signals. The input signals from the controls can be applied to the cells in an array in desired patterns, including, but not limited to, all or nothing patterns in which all of the cells, such as a plurality of variable stiffness robotic material cells 100, are actuated or none of the cells are actuated, pixel based patterns in which each individual cell, such as a pixel, is actuated, or channel based patterns in which the cells are grouped in channels, i.e., along straight line or other paths, for common actuation or non-actuation. In one example, the computer input signal allows for tuning of the variable stiffness actuator mechanism 102. For example, the current and/or voltage of the variable stiffness robotic material cell 100 are tunable to affect the output force and thus the rigidity of the variable stiffness robotic material cell 100.

The sensor 104 is generally a sensor or measuring element that can measure output or other environmental data, which can be fed back into an open-loop or closed-loop system, such as the computation component 106, for self-correction operations, or fed into data storage. Examples of suitable sensors 104 include, but are not limited to, touch sensors, thermal sensors, impedance sensors, pressure sensors, flow sensors, strain sensors, accelerometers, bend sensors, visual sensors such as optical coherence topology sensors and narrow band imaging sensors, optical sensors and chemical sensors.

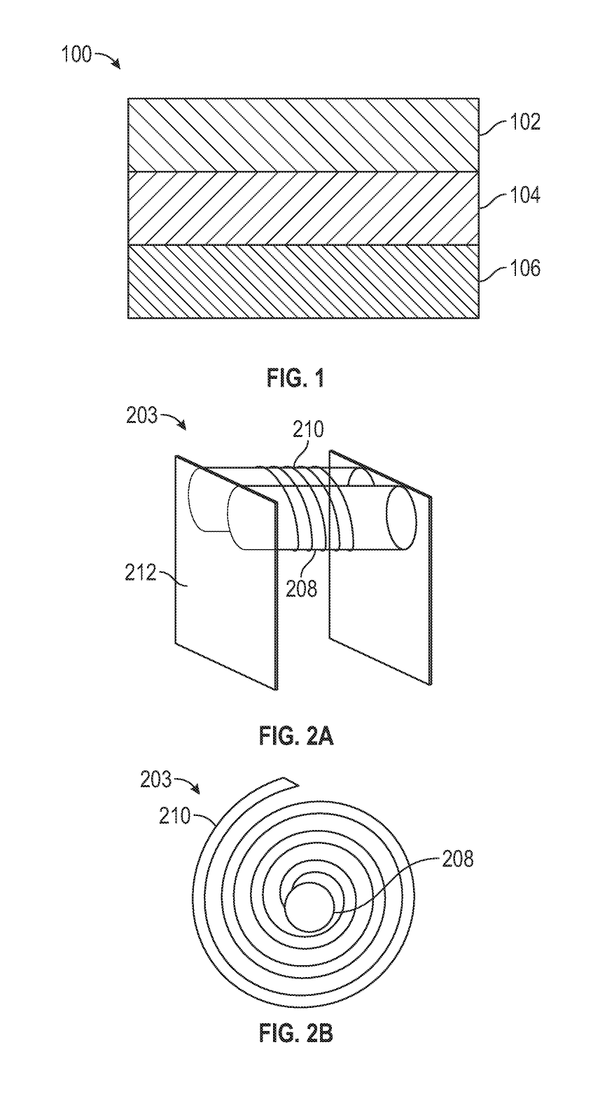

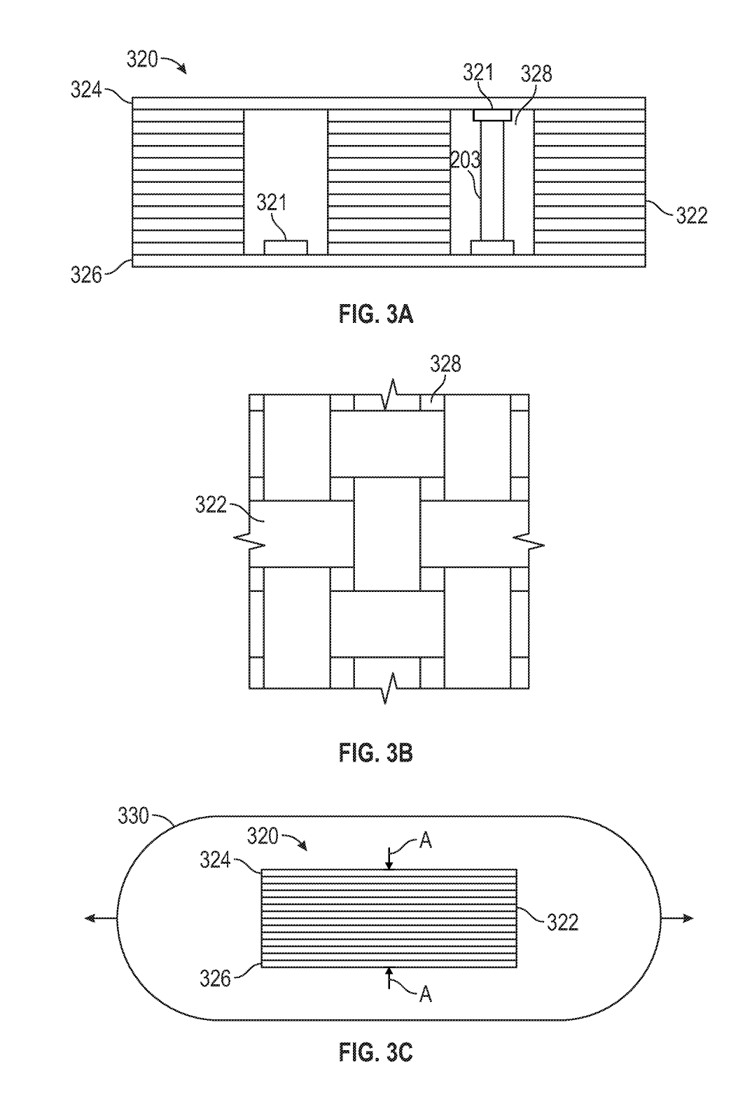

In one embodiment, the variable stiffness actuator mechanism 102 includes an electropermanent magnet and a plurality of jamming layers disposed between two flanges. FIGS. 2A-2B depict electropermanent magnets 203 according to embodiments disclosed herein. FIGS. 3A-3C depict a plurality of jamming layers 320 according to embodiments described herein.

As shown in FIG. 2A, the electropermanent magnet 203 includes a pair of electromagnetic rods 208 (typically a high coercivity rod and a low coercivity rod) having wiring 210, such as copper wiring, wound there around, and the magnets are held in place by metal sheets 212 on the opposed ends thereof. In one embodiment, the pair of electromagnetic rods 208 includes an AlNiCo V rod and a NdFeB 40 rod, which are epoxied to one another in parallel and capped on either end by ferromagnetic rectangular sheets of metal acting as pole pieces, where the width of the pole pieces is equivalent to two times the diameter of the rods, the length is of a comparable dimension, and the thickness is of a much smaller dimension. One or more wires 210 are wound around the electromagnetic rods 208 in a coil shape. Flowing a current through the wires 210 creates a magnetic field about the long axis of the coil, and the magnitude of the magnetic field, and associated force, are a function of the direction of current flow, the voltage and current value on and flowing through the wire of the coil, and the number of turns and dimensions of the wire 210. Here, both ends of the wire(s) 210 of the coil are connected to a control circuit that provides short current pulses that switch the electropermanent magnet 203 between the “on” and “off” state. When power is applied to the coil of the electropermanent magnet 203, the electropermanent magnet 203 is in the “off” state if the poles of the low and high coercivity magnets thereof are misaligned such that their magnetic fields point in opposite directions, effectively creating a closed magnetic circuit with the pole pieces. The assembly is in the “on” state if the poles of the low and high coercivity magnets are aligned such that their magnetic fields point in the same direction, effectively creating an open magnetic circuit with the pole pieces. As shown in FIG. 2B, the electropermanent magnet 203 is flat and includes, for example, low coercivity magnetic material 208 surrounded by concentrically wound wire 210. By powering the wire 210 to flow current therethrough, depending on the direction of current flow, the magnetic pole of at least one of the electromagnetic rods 208 can be flipped, to cause the magnetic field to form at least in part exteriorly of the ferromagnetic pole plates, or to form a closed magnetic circuit including the electromagnetic rods 208 and the pole pieces 212 such that no, or only a very low, magnetic field is present exteriorly of the rods 208 and pole pieces 212.

The plurality of jamming layers 320 is generally a stack of two or more thin sheets 322 (twelve are shown as an example) disposed between a top compression sheet 324 and a bottom compression sheet 326. As shown in the embodiment of FIG. 3A, the plurality of jamming layers 320 includes one or more actuator channels 328 therein, in which a variable stiffness actuator, such as the electropermanent magnet 203, is positioned. A ferromagnetic sheet 321, such as a strike plate, is positioned at the bottom of each of the one or more actuator channels 328 and is attached to a first surface of the bottom compression sheet 326, i.e., the surface of the bottom compression sheet 326 facing the thin sheets 322. A second ferromagnetic sheet 321 is positioned at the top of each of the one or more actuator channels 328 and is attached to a second surface of the top compression sheet 324, i.e., on the surface of the top compression sheet 324 facing the thin sheets 322. When the electropermanent magnet 203 is controlled to locate the magnetic field exteriorly of the pole pieces 212 and the rods 208, the ferromagnetic plates are magnetically attracted toward the adjacent end of the electropermanent magnet 203 thereto, and thus the sheets 321 squeeze together the stack of thin sheets 322. When the electropermanent magnet 203 is controlled to create a closed magnetic circuit therein, the sheets 321 are not magnetically attracted to the adjacent end of the electropermanent magnet 203, and squeezing of the thin sheets 322 does not occur. Alternatively, the top and bottom 324, 326 compression sheets may, themselves, be composed of a magnetizeable material, in whole or part.

Each thin sheet 322 can be any material that is greater in length and width than in thickness. Each thin sheet 322 is generally any suitable material having both high surface friction and also high elasticity. The stiffness of the stack of sheets is governed by the equation F=P*μ*N*A where F is the force required to bend the stack, P is the pressure applied perpendicular to the surface of the sheets, μ is the coefficient of friction of the surface of the thin sheet, N is the number of sheets in the stack undergoing the applied pressure P, and A is the surface area of contact between individual thin sheets undergoing the applied pressure P. In one embodiment, each thin sheet 322 is a material having three-dimensional, or other hierarchical structures, therein to increase the variable stiffness by increasing the surface area of contact (A) between each of the thin sheets 322. In another embodiment, each sheet is made of high-friction Tyvek having a thickness of between about 0.10 and about 0.20 millimeters (mm), such as about 0.15 millimeters.

The configuration of the one or more actuator channels 328 is predetermined based on the rigidity requirements of the specific application. For example, the configuration of the one or more actuator channels 328 and the actuators disposed therein, is predetermined based on information collected from sensors, such as a sensor 104, in the variable stiffness robotic material cell 100. The one or more actuator channels 328 are generally any suitable shape, including, but not limited to, rectangular or cylindrical channels. The position of each of the one or more actuator channels 328 is generally configured to provide a predetermined stiffness in the plurality of jamming layers 320 and the overall stack thereof. In one example, the one or more actuator channels 328 are positioned close to one another in order to reduce pressure loss in the plurality of jamming layers 320 between actuators positioned in adjacent actuator channels 328.

In operation, force generation from activation of the electropermanent magnet 203 pulls the top compression sheet 324 and the bottom compression sheet 326 towards the middle of the stack of two or more thin sheets 322, exerting a pressure perpendicular to the surface of the stack of two or more thin sheets 322. This force causes the rigidity of the plurality of jamming layers 320 to increase and increases the stiffness of the plurality of jamming layers.

In further embodiments, by selectively powering some but not all of the electropermanent magnets 203, from 1% to 100% of them, the stiffness of the overall stack can be varied.

As shown in the embodiment of FIG. 3B, the two or more thin sheets 322 are interwoven and include one or more actuator channels 328 at the interstitial spaces between the interwoven thin sheets 322 of the plurality of jamming layers 320.

As shown in the embodiment of FIG. 3C, the plurality of jamming layers 320 do not include one or more actuator channels. During operation of the embodiment of FIG. 3B, the plurality of jamming layers are acted upon by an indirect force (shown with arrows A) to increase rigidity, such as by pulling the ends of an enclosure 330 to pull the sidewalls thereof together and against the opposed sides of the stack of thin sheets 322, or by pulling a vacuum in the enclosure to create a sub-atmospheric pressure therein, such that the surfaces of the enclosure are drawn inwardly against the opposed sides of the stack of thin sheets 322, with a pressure equal to the difference between the ambient atmospheric pressure and the lower vacuum pressure in the enclosure.

In another embodiment, the variable stiffness actuator mechanism includes an electropermanent magnet disposed between a first compression sheet and a second compression sheet. At least one of the first compression sheet or the second compression sheet includes a strike plate. Unlike the embodiments depicted in FIGS. 3A-3C, the variable stiffness actuator mechanism does not include a plurality of jamming layers between the first compression sheet and the second compression sheet. In operation, when activated the electropermanent magnet interacts with the strike plate to increase the rigidity of the variable stiffness robotic material cell.

In another embodiment, the two adjacent compression sheets have planar electromagnet or planar electropermanent magnet coils printed onto the sheet surfaces. When activated, these planar, circuit-printed magnetic actuation mechanisms are pulled towards ferromagnetic regions on the portion(s) of the other compression sheet directly opposite the magnet coils.

The robotic material cells, such as variable stiffness robotic material cells, described herein are useful for a variety of applications. The disclosed robotic material cells are useful for medical devices such as endoscopes, implantables, surgical robotics, exoskeletons, splits, casts, braces, orthodontics, catheters, moldable cosmetic implants, oral appliances and sleep apnea implants. The disclosed variable stiffness robotic material cells are useful for health and fitness applications such as weights, resistance clothing, resistance equipment, rehabilitation equipment, and adjustable stiffness beds. The disclosed variable stiffness robotic material cells are useful for aerospace applications such as wings, air foils, dampening systems, structural systems, landing systems, solar panel systems and deployable systems in space. The disclosed variable stiffness robotic material cells are useful for energy applications. The disclosed variable stiffness robotic material cells are useful for defense devices such as body armor, vehicles, tire systems, shelter, and moldable exteriors. The disclosed variable stiffness robotic material cells are useful for automotive applications such as seat and whiplash support, moldable exteriors, and tires.

According to embodiments of the present disclosure, a plurality of variable stiffness robotic material cells, such as variable stiffness robotic material cells 100, are combined in any suitable configuration. For example, the variable stiffness robotic material cells can be combined periodically or aperiodically to form a hollow or solid tube, which may be used as a sleeve or overtube that surrounds one or more channels for various applications. In the example of a hollow tube, the hollow tube may be a single-channel or multiple-channel tube, depending on the operation for which the tube will be used. For example, the variable stiffness robotic material cells can be configured to form reinforcement tubing for various devices, such as a variable stiffness endoscopic sleeve with a working channel that uses layer jamming actuated by electropermanent magnets within the endoscope tubing as the shape-locking mechanism.

In one embodiment, an overtube or sleeve that may fit over existing endoscopes, providing the user with flexibility to navigate difficult to reach areas inside a patient, is disclosed. The device may change to a rigid state on user activation of the actuation elements, for example the electropermanent magnets, giving the user the ability to perform precise manipulation of tissue. The sleeve is preferably made up of an inner layer located adjacent to the endoscope, a middle actuating layer, and a third outer layer for insulation. The actuating layer is coiled around the innermost layer, and can be activated to make the whole sleeve transform from flexible to rigid. The actuating mechanism can incorporate magnets, piezoelectrics, ionic polymers, shape memory alloys and/or microelectromechanical systems (MEMs) to actuate. When the actuating layer is activated, it reversibly converts the state of the sleeve from flexible to rigid.

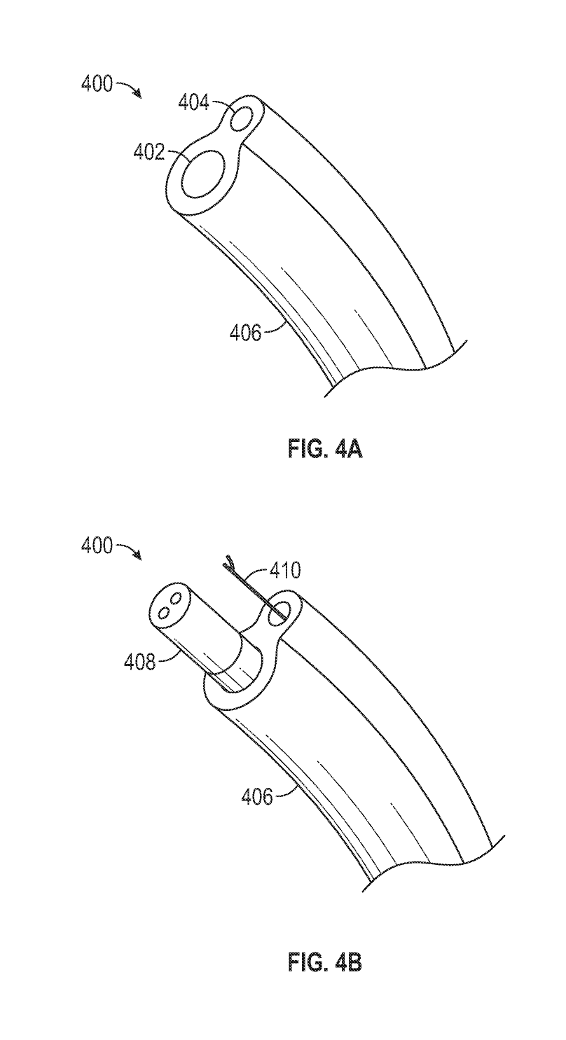

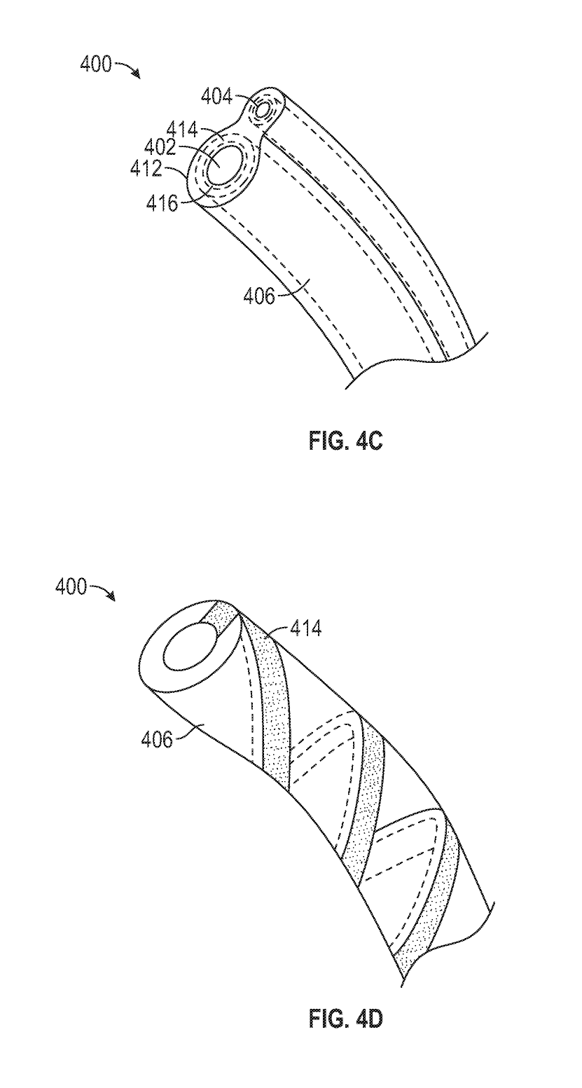

FIGS. 4A-4D depict an endoscope overtube 400, according to embodiments disclosed herein. As shown in FIG. 4A, the endoscope overtube 400 generally includes a first channel 402 and a second channel 404 defined by a body, shown as a surrounding sleeve 406. In one embodiment, the first channel 402 is an endoscope channel having an endoscope 408 therein and the second channel 404 is a working channel having a tool 410, such as a dilation balloon assembly, a needle, forceps, or a catheter fitted at the distal end for any type of ablation included radiofrequency, cryoablation, microwave, laser, ultrasound, and electroporation delivery therein, as shown in FIG. 4B. The sleeve 406 generally includes a plurality of layers at least one of which is an actuation layer.

As shown in FIG. 4C, the sleeve 406 includes an actuation layer 414 disposed between a first polymer layer 412 and a second polymer layer 416. The actuation layer 414 comprises a plurality of robotic material cells, such as variable stiffness robotic material cells 100. The first polymer layer 412 and the second polymer layer 416 are generally any suitable polymer materials to provide insulation and/or structure to the endoscope overtube 400.

The amount and configuration of the actuation layer 414 is determined based on the desired level of stiffness for the procedure to be conducted. Information regarding the desired level of stiffness for the procedure is generally predetermined, for example, based on information collected by sensors, such as sensor 104, in the variable stiffness robotic material cells, such as the variable stiffness robotic material cells 100. For example, the potential stiffness amount and configuration of the actuation layer 414 is selected to transmit the force and the torque at the tip of the sleeve 406 that the physician applies at the base of the tool having the sleeve 406. In the example of a sleep apnea procedure, the amount and configuration of the actuation layer 414 is selected to provide enough rigidity to the sleeve 406 such that the tool 410 in the working channel 404 can penetrate a patient's tissue in order to treat sleep apnea. In one embodiment, the actuation layer 414 extends the entire length of the sleeve 406. In another embodiment, the actuation layer 414 only comprises a portion of sleeve 406, such as at the tip thereof. The actuation layer 414 can be in the shape of a band extending along the length of the sleeve 406, a spiral coiled around the sleeve 406 as shown in FIG. 4D, or a braid around the sleeve 406. In operation, the rigidity of the actuation layer 414 is adjusted according to user input, as described above. When the actuation layer 414 is activated, the actuation layer 414 becomes rigid. This in turn forces the entire endoscope overtube 400 to become stiffer. For example, in operation, a physician generally presses a button, which, through a printed circuit board (PCB), activates the variable stiffness actuation mechanism and causes the EPMs to bias the strike plates to activate layer jamming, thus resulting in controllable variable stiffness on-demand as desired by the physician.

FIGS. 4A-4D depict a sleeve-shaped configuration of a plurality of variable stiffness robotic material cells. Other suitable configurations of a plurality of variable stiffness robotic material cells include, but are not limited to, fabric-like configurations and spherical configurations.

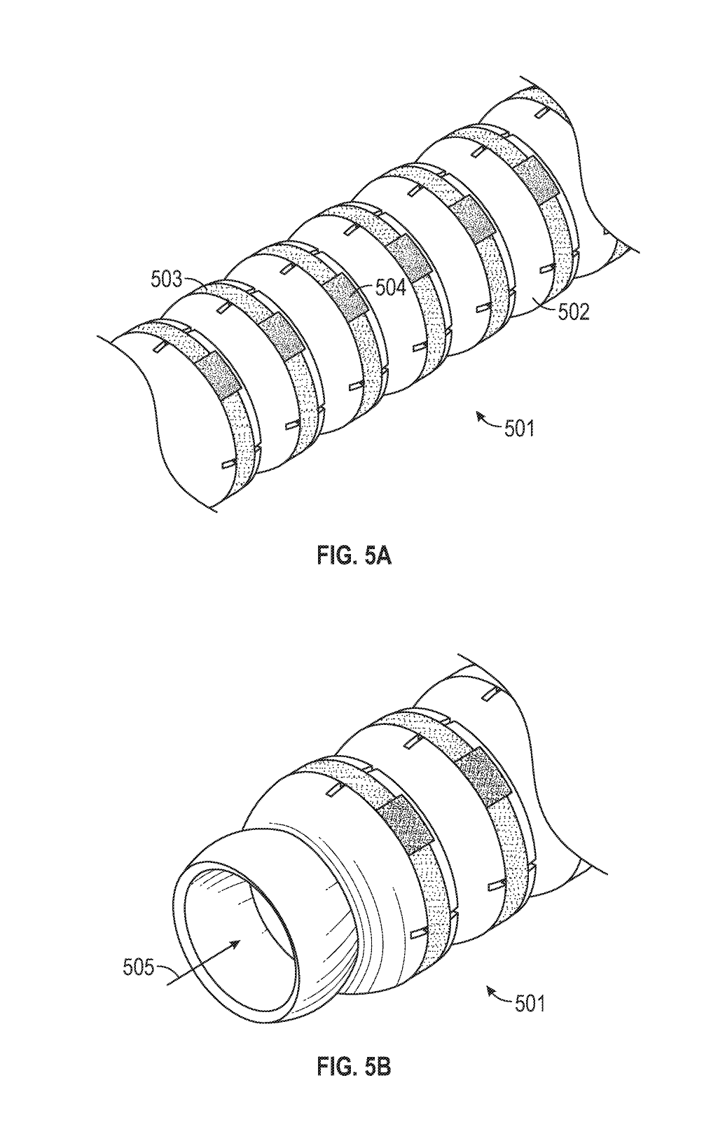

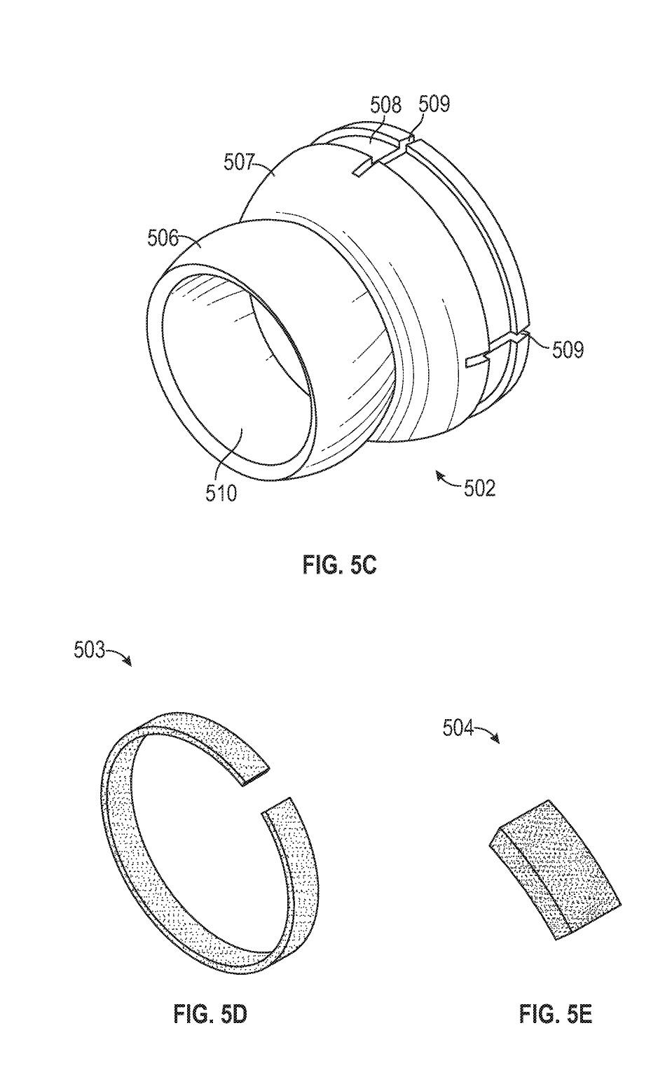

FIGS. 5A-5E depict a shape-locking overtube assembly 1 according to embodiments disclosed herein. The sleeve is preferably made up of numerous individual modules that may fit together using ball-and-socket joints. The socket portion of each module, when actuated, preferably cinches down on the ball portion of an adjacent module, effectively changing the state of the entire sleeve from flexible to rigid. The cinching of each socket may be accomplished via a device that actuates a ring around the socket portion of a module, effectively reducing the circumference of the socket portion. This device and ring mechanism may incorporate magnets, piezoelectrics, ionic polymers, shape memory alloys and/or microelectromechanical systems (MEMS) to actuate.

The individual modules that make up the sleeve are preferably designed such that ball-and-socket joints associated with the modules provide adequate mobility to allow the sleeve flexibility when not actuated to become rigid.

The center of each module is preferably hollow to allow for the passage of an instrument there through. The hollow channel through the module may be tapered at the ball end to prevent pinching of the instrument during movement.

The socket portion of each module may include relief notches to allow for the ball portion of the subsequent module to seat properly during assembly as well as to allow for adequate inward deflection during cinching.

The socket portion may have an indentation circumferentially surrounding the entire module to allow for the cinching mechanism to fit. The inward deflection of the socket during cinching will preferably reduce the circumference of the socket.

The cinching action described will cause the socket portion to put pressure on the ball portion of the subsequent module no matter what orientation it is in, effectively holding it in place. Every module associated with the sleeve performing this action simultaneously will cause the entire sleeve to become rigid.

FIG. 5A shows a span of the sleeve assembly including the connected modules and cinching mechanisms.

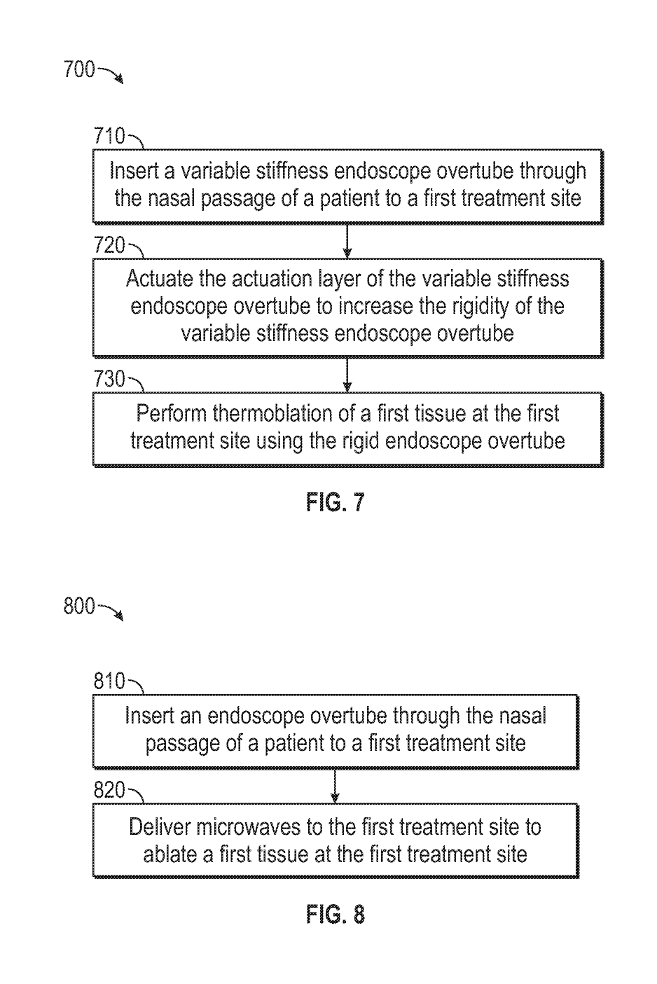

FIG. 5B shows a hollow channel through the sleeve of FIG. 7A and where the instrument may be inserted.

FIG. 5C is a perspective view of a module that makes up a sleeve.

FIG. 5D is a perspective view of a ring that may be used as a cinching mechanism.

FIG. 5E is a perspective view of a cinching member that makes up the cinching mechanism.

FIG. 5A shows a span of a sleeve 501 that is made up of a plurality of interconnected modules 502. Each module 502 contains a slotted ring 503 having a gap and cinching member 504 that preferably makes up the cinching mechanism seated in the indentation 508 of the socket 7 (FIG. 5C). The ball portion 506 of each module 502 can rotate freely inside the socket portion 507 of each preceding module 502 when the cinching mechanisms are not engaged, allowing for flexibility of the sleeve 501 over its length. When the cinching mechanisms are engaged, they pull together the ends of the ring across the gap therein, and thereby cause the ring diameter to be reduced to squeeze and thereby cinch down the socket portion 507 of each module 502, holding the ball portion 506 of the subsequent module 502 in its current orientation, effectively making the entire sleeve 501 rigid.

FIG. 5B shows the proximal end of the sleeve assembly 501. Each module 502 may contain a hollow channel 510 for the passage of an instrument 505. The sleeve 501 would then provide the instrument with flexibility or rigidity at the user's discretion.

FIG. 5C illustrates an embodiment of module 502. The ball portion 506 preferably fits inside the socket portion 507 of the preceding module 502, allowing for rotation. The center of each module 502 may contain a hollow channel 510 for the insertion/passage of an instrument 505. The indentation 508 preferably allows for the cinching mechanism to fit within the module 502. Upon cinching of the socket portion 507, the relief notches 509 allow for further inward deflection of the socket portion 507 to adequately squeeze the ball portion 506 of the adjacent module.

FIG. 5D illustrates a ring 503 that makes up the cinching mechanism.

FIG. 5E illustrates a cinching member 504 that makes up the cinching mechanism. The cinching member 504 may function by either drawing in or transferring energy through the ring 503 such that the ring tightens about the socket portion 507 of each module 502. The cinching member 504 may involve the usage of magnets, piezoelectrics, ionic polymers, shape memory alloys and/or microelectromechanical systems (MEMS) to actuate.

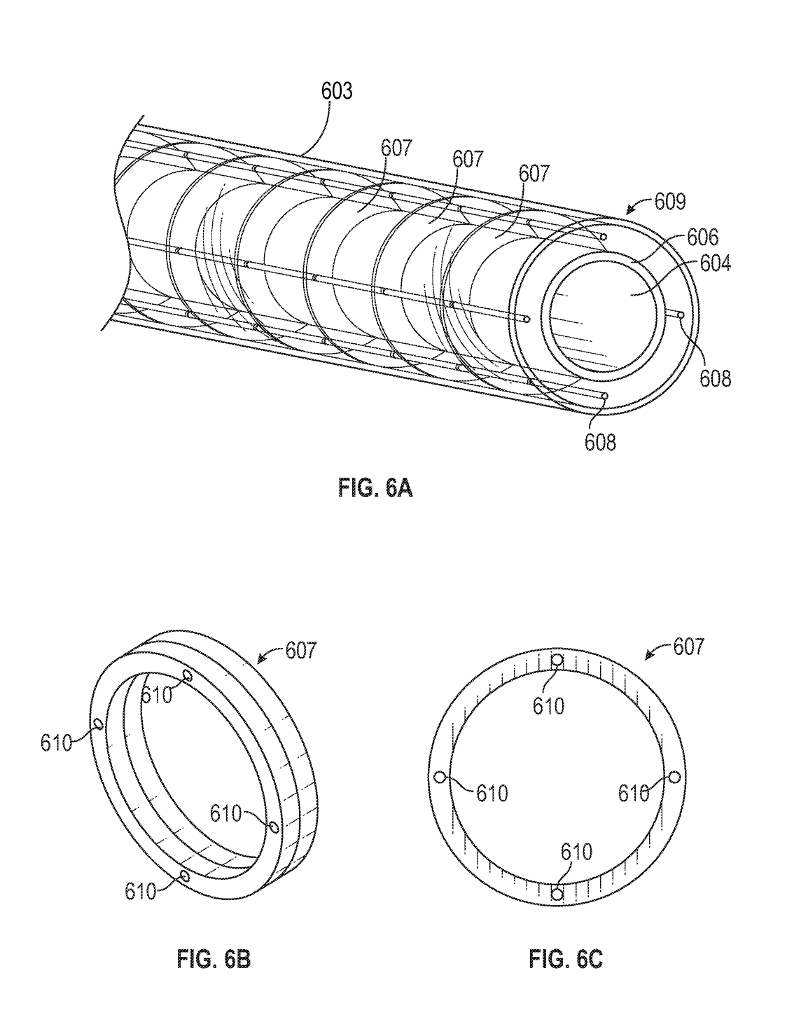

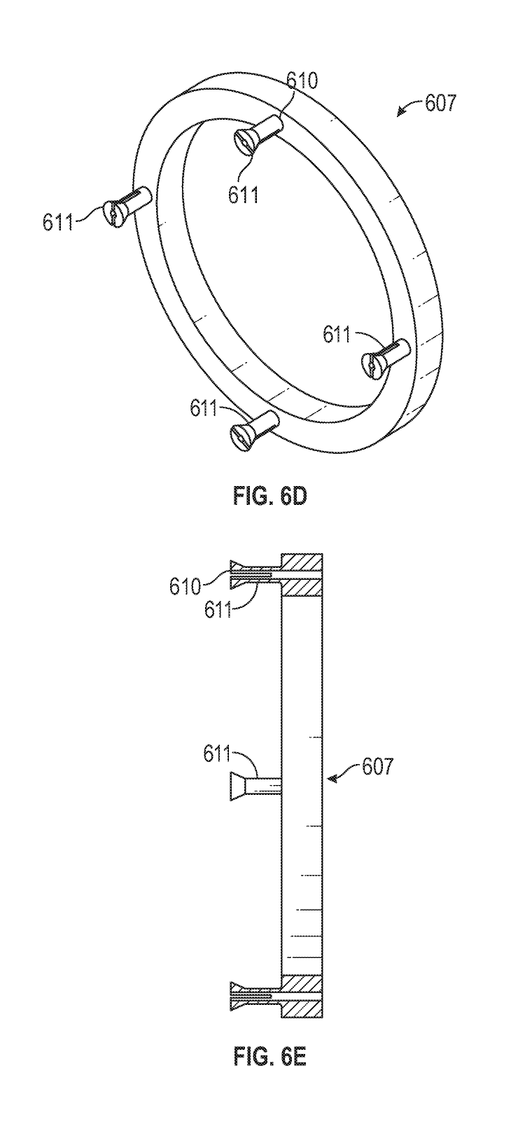

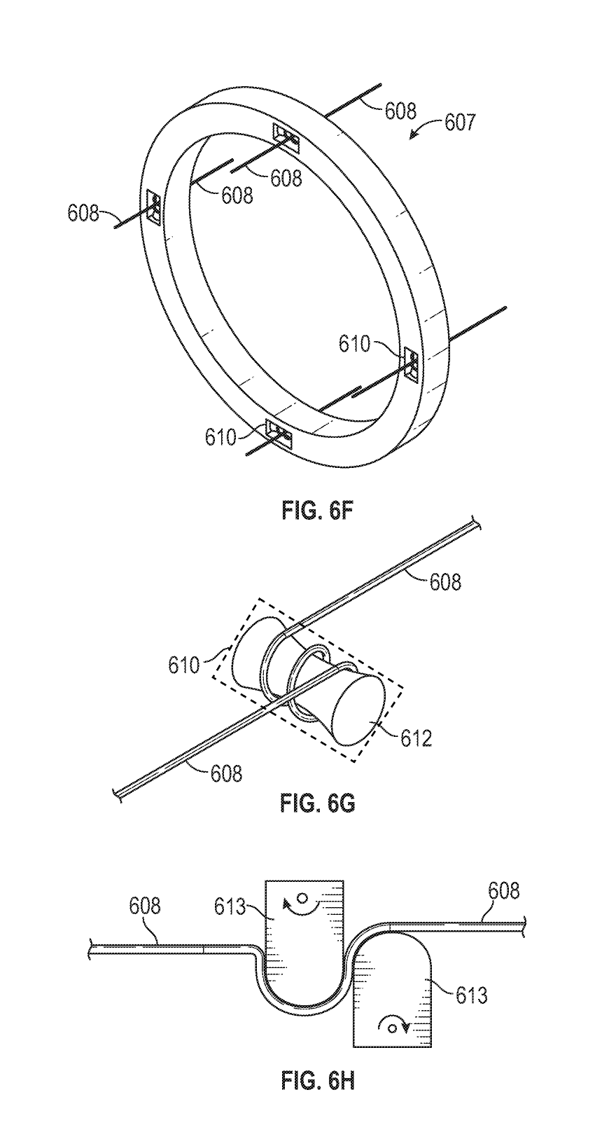

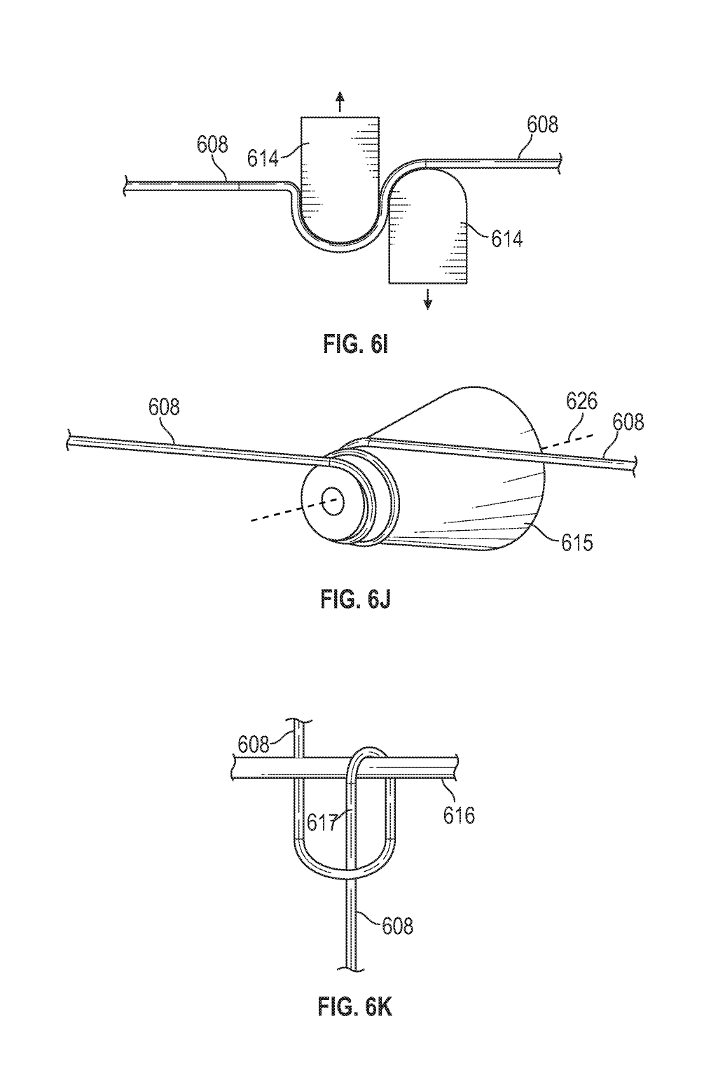

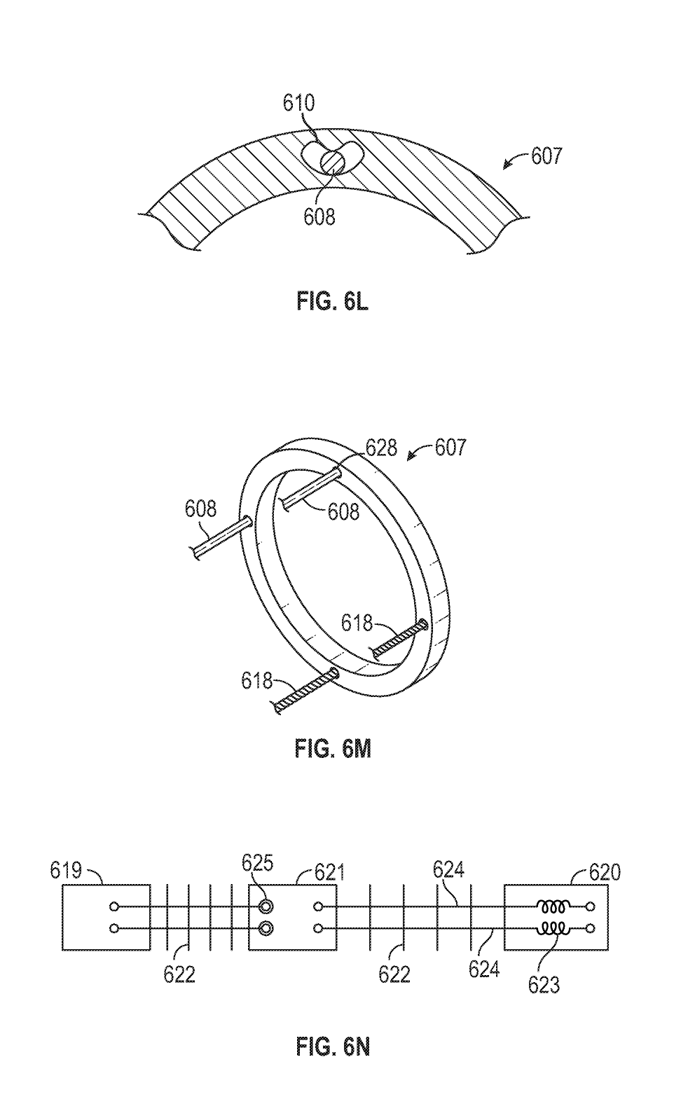

FIGS. 6A-6N depict a shape-locking overtube assembly according to embodiments disclosed herein. Different segments along the working shaft length of the shape-locking overtube can be activated separately, thereby providing modularity for navigating around obstacles. For this reason, electrically controlled actuators are placed locally along the shaft length to perform the task of locally locking the overtube shape, rather than inside a handle attached to the overtube. However, the attached handle may incorporate additional actuators if deemed necessary by stiffness requirements or to provide steerability by selectively tensioning lines.

The distal shaft of the shape-locking overtube consists of an assembly of multiple components. An inner tube of reinforced polymer provides the main lumen for the insertion of a tool such as a scope and to provide structural stability to counteract line tension. Rigid rings are bonded to the inner tube. Rings provide attachment for local actuators and provide additional torsional stiffness. Approximately three to eight lines traverse the length of the distal working shaft of the shape-locking tube passing through openings in the rings. The lines have very high elastic modulus in tension but have very low flexural stiffness, acting like tendons. All lines are kept in tension at all times (no slack), for example with the use of torsional springs with pulleys that are incorporated into joints and terminals, the proximal terminal being incorporated into the handle, or by the use of tensioning devices in between the rings, or by incorporating automatic tensioning into actuators placed on the rings. An outer tube of low stiffness provides an outer lining or shroud over the inner components of the shape-locking overtube.

Instead of rings, one may use linkages of other shapes, such as a starfish-like shape with each arm holding one line, or spokes emanating from a hub where one or more spokes are connected to actuators within which lines are threaded. Instead of lines, which are thought to be approximately circular or elliptical in cross-section, one may use tapes, which are rectangular in cross-section with a large aspect ratio, or belts within which grooves are embedded or cogs are built that mesh with cogs on sprockets, or chains whose links mesh with cogs on sprockets, which are connected to actuators.

To produce shape-locking, a button located on the overtube handle is pushed, activating the local actuators located in the rings such that they hold the tensioned lines in place, i.e. prevent their axial motion relative to the rings in the active state. To return to the flexible, inactive state of the overtube, the button on the overtube handle is again pushed and the actuators release the tension on the lines such that they are free to move axially relative the rings during bending.

Various principles may be used to hold and release the lines during shape-locking and unlocking at the rings located along the length of the distal shaft. All principles of operation are bound by the requirement that a single ring/actuation unit should not hold all the tension on a line. The load of a line should be distributed across multiple rings along the length of the tube, thus reducing the risk of failure and lowering the holding requirements for single actuators. The choice of the principle to be used depends on the choice of actuation. Actuation mechanisms could include but are not limited to piezoelectric, electromagnetic, electro-permanent magnetic, electroactive polymers, and shape memory alloys.

Since each ring may be actuated independent of the others, actuating a group of adjacent rings while leaving others inactive results in modular shape locking, i.e., shape locking a segment of the overall length of the shape locking overtube assembly. Modularity may also be improved by reducing ring spacing and increasing inner tube stiffness, either by change of materials, dimensions or structure, thereby allowing higher line tension in the active state.

A flexible segment of the device consists of an assembly of rings, lines and tubes. A segment may be attached to a rigid joint or a terminal at either end. A terminal has only one flexible segment attached to it. A joint has two or more flexible segments attached to it. For the overtube described above, its handle and its distal end incorporate terminals. Within a joint or a terminal, a line may be terminated with a fixed attachment point, or with a linear spring, or with a torsional spring and spool assembly, for the purpose of maintaining positive tension. Alternatively, a line may terminate at a rotational actuator which removes slack and maintains tension.

FIG. 6A shows a close-up of the assembly of one segment of the distal shaft of the overtube

FIG. 6B is a view of an individual ring assembly that uses a shearing mechanism to lock the lines in place.

FIG. 6C is another view of an individual ring assembly with adjacent shearing rings counter-rotated in the “locked” position

FIG. 6D is a spanned view of an individual ring assembly that uses a collet mechanism to lock the lines in place.

FIG. 6E is a side view of an individual ring assembly that uses a collet mechanism to lock the lines in place.

FIG. 6F is a spanned view of an individual ring assembly that uses a capstan mechanism to lock the lines in place.

FIG. 6G is a spanned view of an individual spinning capstan used to lock the lines in place in a capstan ring assembly.

FIG. 6H is a view of an individual rotating levered capstan used to lock the lines in place in a capstan ring assembly.

FIG. 6I is a view of an individual linear levered capstan used to lock the lines in place in a capstan ring assembly.

FIG. 6J is a spanned view of an individual tapered spinning capstan used to lock the lines in place in a capstan ring assembly.

FIG. 6K is a view of an individual knot mechanism used to lock the lines in place in a knot-ring assembly.

FIG. 6L is a view of an individual ring made from shape-memory alloy shown in its warped shape.

FIG. 6M is a view of one section of a shape-memory braided line in its expanded shape.

FIG. 6N is a skeleton drawing of an elongate steerable device with shape locking capability showing two rigid terminals and one rigid joint connecting two flexible segments.

FIG. 6A shows the shape-locking overtube assembly contained within shaft 603. The lumen 604 is formed by an inner tube 606 constructed of a flexible material resistant to kinking and buckling such as a polymer matrix reinforced with steel or Nitinol or carbon-fiber braid or coil. Distributed along the length of, and attached to the outer surface of, the inner tube 606 are actuator rings 607 through which the tensioned lines 608 that run along the length of the shaft 603 extend. The rings 607 contain actuators that have the ability to hold and release the tensioned lines 608 for the purpose of locking the shaft 603 at or in a given geometric configuration. An outer polymer shell 609 surrounds and protects the entire assembly, creating the outer surface of the shaft 603.

FIG. 6B and FIG. 6C show an example of a single actuator ring assembly 607 that uses a shearing mechanism to achieve the holding and releasing of the lines 608. Channels 610 extending through the ring assembly 607 in the local longitudinal direction of the shaft 603 allow for the passage of the lines 608 through the ring assembly 607. A rotation of the two rings of the assembly 607 relative to one another (FIG. 6D) as caused by the activation of the embedded actuator leads to a circumferential rotation of at least one portion of a ring assembly relative to the other portion thereof, resulting in a reduction of the cross section of the channels 610 of the lines 608 to allow portions of the inner surfaces of the channels to grip or grasp the line extending therethrough, in turn locking the shape of that segment of the shaft 603 with respect to the tensioned lines 608.

FIG. 6D and FIG. 6E show an example of an alternative single actuator ring assembly 607 that uses a collet mechanism to achieve the holding and releasing of the lines 608. Hollow collets 611 extend from one side of the ring, the hollow interiors of which lead directly into the openings of the channels 610 in the ring assembly 607 and thereby allow for the passage of the lines 608 through the channels in the ring assembly 607. A collet 611 consists of a material or materials that are constructed so as to allow the inner diameter of the collet 611 to be constricted (shrunk or reduced in cross section) or dilated (enlarged in cross section. The activation of the embedded actuator in the ring assembly 607 leads to tightening of the collets 611 around the lines 608, in turn locking the shape of that segment of the shaft 603 with respect to the tensioned lines 608.

FIG. 6F and FIG. 6G show an example of a single actuator ring assembly 607 that uses a capstan mechanism to achieve the holding and releasing of the lines 608. Channels 610 in the ring assembly 607 allow for the passage of the lines 608 through the ring assembly 607. A capstan 612 consists of a material or materials that are constructed so as to leverage the increased surface area of contact between the line 608 and capstan 612 as a means of holding the line from moving axially through the ring assembly 607 as a result of the friction between the capstan surface and the line, which is proportional to line tension, because spring tension pulls the line into contact with the contoured cylindrical surface of the capstan. The activation of the embedded actuator in the ring assembly 607 locks the capstan 612 against rotation and, as the capstan grips the line 608 wrapped around it, locking of the capstan against rotation locks the ring in place with respect to the line, therefore locking in place the local shape of that segment of the shaft 603. In the off-state, the capstan 612 is free to spin on its central axis, which allows the contoured surface to rotate about that axis, and thus allows the line 608 to move relatively freely in the local longitudinal direction of the shaft 602.

FIG. 6H shows a rotating lever capstan 613 mechanism that can be used to hold the lines 608 to prevent them from moving through the channels 610 in ring assembly 607 in the local longitudinal direction of the shaft 603. The rotating levers 613 provide a surface area within the channels 610 for contact between the line 608 and the rotating levers 613. When the levers 613 are positioned for free motion between the rings 607 and lines 608, there is minimal to no contact between the line 608 and adjacent surfaces of the levers 613, providing a sufficiently sized opening for free travel of the line 608 and ring 607 with respect to one another in the local longitudinal direction of the shaft 603. The activation of the embedded actuator in the ring assembly 607 causes the rotating lever capstan 613 to swing about the axis of rotation such that the flattened end surfaces of the levers are vertically facing opposite directions, in turn causing a rounded surface of the levers 607 to engage the line 608 extending through the channel 610 to push adjacent portions of the line in opposite directions perpendicular to the local longitudinal direction of the shaft 602. As a result, the line 608 is pinched between adjacent surfaces of the levers 607, thereby pinching the line 608 therebetween and locking that segment of the shaft 603 to the lines 608. In the off-state, as the levers 613 are horizontally facing outward as shown in dashed line outline in FIG. 6H, they allow the line 608 to move freely within the channel 610.

FIG. 6I shows a linear pinch capstan 614 mechanism that can be used to hold the lines 608 from moving axially through the ring assembly 607. The linear pinches 614 create a surface area of contact between the line 608 and linear pinches 614 as a means of holding the line from moving axially through the ring assembly 607. The activation of the embedded actuator in the ring assembly 607 leads the linear pinches 614 to move linearly across the intended path of the line 608 in the channel 610 through the ring 607, such that the pinches are located adjacent to each other in the local longitudinal direction of the shaft 603, in turn pinching the line 608 and locking that segment of the shaft 603 in the local longitudinal direction of the shaft 603. In the off-state, the levers 614 are non-adjacent 603 in the local longitudinal direction of the shaft 603 and spaced, in a direction perpendicular to the line 608 path in the local longitudinal direction of the shaft 603, allowing the lines 608 to move freely in the channels 610.

FIG. 6J shows a tapered (truncated cone) rotatable capstan 615 mechanism that can be used to hold the lines 608 from moving through the channels 610 in the ring assembly 607 in the local longitudinal direction of the shaft 603. The tapered rotatable capstan 615 creates a surface area of contact between the line 608 and tapered surface of the rotatable capstan 615 as a mechanism for holding the line from moving axially through the ring assembly 607. The activation of the embedded actuator in the ring assembly 607 causes the tapered spinning capstan 615 to be fixed against rotation in the channel 610, and the friction between the line 608 and the tapered surface of the capstan 615 causes line 608 wrapped around it to become fixed against movement in the local longitudinal direction of the shaft 603, in turn locking that segment of the shaft 3 in place with respect to the line 608. In the off-state, the tapered rotatable capstan 615 is free to rotate about its central axis 626 allowing the line 608 to move freely. Due to its tapered shape, linear movement along the central axis 626 also allows the tapered spinning capstan 615 to modulate the amount of tension on the line 608.

FIG. 6K shows a knot 617 mechanism that can be used to prevent the lines 608 from moving axially through the ring assembly 607 in the local longitudinal direction of the shaft 603. The knot 617 creates a surface area of contact between the line 608 and rod 616 as a means of holding the line from moving axially through the ring assembly 607. The activation of the embedded actuator in the ring assembly 607 leads the line 608 to become knotted 617, in turn pinching the line 608 against the rod 616 and locking that segment of the shaft 603 to the line 608. In the off-state, the line 608 moves freely through the channel 610.

FIG. 6L shows an opening or channel 610 in a ring 607 made from a shape-memory alloy, such as Nitinol, in its gripping or pinching state, which is firmly holding a line 608 made of a harder material, such as steel to prevent the ring 607 from moving along the length of the line 607 in the local longitudinal direction of the shaft 603. The ring 607 is configured, so that in its free, higher temperature state, it grips the line 608. In its low temperature state, the phase change material of the ring 607 is deformed from that of the high temperature state, wherein the channel 610 is enlarged from that of the high temperature state and the line 608 can move freely in the channel. Upon application of heat, the temperature rise transforms the material to the stiffer high temperature phase and reverts it to the memory shape, thereby causing the ring 607 to grip the line 608 extending through the channel 610. Upon cooling, the material returns to the phase where the opening area of the channel 610 is increased. The heat source may be resistive heating in wires assembled into the rings 607 for that specific purpose. In another embodiment, the heat may be a by-product of electrical energy transmission within a cable or waveguide placed in a working channel, such as those carrying microwave energy.

FIG. 6M shows a line 608 made from a shape memory alloy placed coaxially into a rigid, hollow cylinder 628, which is attached to a ring 607. The line can be in the form of a single strand structure 608 that changes its diameter when heated, or a woven, multi-strand structure 618 that experiences an overall change in diameter due to a change in the conformation thereof upon being heated. The cylinder 628 is made from a material with low thermal expansion coefficient. At low temperature, the line structure has a smaller diameter and the line moves freely within the cylinder in the longitudinal direction thereof. Upon heating of the line 608, the transitioning thereof to a higher temperature and stiffer phase and reverting to the memory shape causes the line diameter to increase (single strand 608) or causes the structure diameter to increase (multi-strand 618), thereby creating friction between itself and the cylinder 628, restricting travel of the line 608 in the longitudinal direction of the cylinder 628.

FIG. 6N is a schematic of an elongate steerable device with shape locking capability showing two rigid terminals 619, 620 and one rigid joint 621 connecting two flexible segments 622. The proximal terminal 620, which may be assembled into the handle of a surgical tool, has spring attachments 623 for the lines 624 which maintain tension, i.e., the ends of the lines 624 proximal to the tool handle (not shown) are spring biased to pull the proximal ends thereof inwardly of the handle and thereby impose tension thereon. The medial joint 621 has fixed attachments for connecting the end of the lines 624 distal to the tool handle thereto on its proximal side, and rotational actuators 625 on its distal side. The proximal ends of a second set of lines are connected to a shaft of a rotational actuator. The actuators 625 have the double purpose of maintaining tension of the lines 624 of the second set of lines 624, and selectively increasing the length of some lines 624 while reducing those of others of the second set of lines 624 for actively bending the distal segment. The distal terminal 619 has fixed attachments for connecting the end of the second set of the lines 624 distal from the medial joint 621 thereto. Changing the length of the lines 624 of the second set of lines 624 is accomplished by selective rotation of the shafts of the actuators 625 about which the proximal ends of the second set of lines 624 are wound. Rotation of a shaft in a first direction unwinds a portion of the line 624 therefrom, thereby increasing the length thereof between the shaft of the actuator 625 and the fixed connection thereof to the distal terminal 619. Rotation of the shaft in a second, opposite, direction decreases the length of the line 624 between the shaft of the actuator 625 and the fixed connection thereof to the distal terminal 619. End-effectors, such as needles, graspers or surgical cutting tools may be included in the distal terminal 619.

In another embodiment, the shape-locking overtube assembly may include a single set of lines, which are fixed in tension over a first length thereof, for example, between the proximal terminal 620 and the medial joint 621, and which are changeable in length between the distal terminal 619 and the medial joint 621.

While the foregoing embodiments contemplate a variable stiffness endoscope overtube, it is also contemplated that the variable stiffness material or mechanism may also be directly incorporated into an endoscope, or other device, itself such that the stiffness of the endoscope or other device itself may be varied as desired.

FIG. 7 is a flow chart of a method 700 according to embodiments disclosed herein. The method 700 is generally a multi-site thermoblation method using a variable stiffness endoscope overtube, such as the variable stiffness endoscope overtubes described herein and shown in FIGS. 4A-4D, FIGS. 5A-5E, and FIGS. 6A-6N, which procedure can be performed in a physician's office. The method 700 begins at operation 710 by inserting the flexible endoscope into the variable stiffness endoscopic overtube when in the flexible state. The endoscope covered in the variable stiffness overtube is inserted through the nasal passage of a patient to a first treatment site. Once the inserted end of the endoscope is positioned at the first treatment site, the actuation layer of the endoscope overtube is actuated to increase the rigidity of the overtube assembly, at operation 720. Next, at operation 730, the physician uses the endoscope held in place by the rigid overtube assembly to perform thermoblation of a first tissue at the first treatment site. It is also contemplated herein, that the end of the endoscope may be actuated away from, and inwardly of, the tip end of the overtube fed into the patient. The method 700 is customizable and is generally repeated to treat any number of sites within the patient. For example, after the thermoblation at the first treatment site, the actuation layer is deactivated to reduce the rigidity of the overtube and increase the flexibility of the overtube assembly. The physician then directs the endoscope and overtube assembly to a second treatment site. Once at the second treatment site, the actuation layer of the overtube assembly is actuated to increase the rigidity of the overtube assembly. Next, the physician uses the endoscope held in place by the rigid overtube to perform thermoblation of a second tissue at the second treatment site.

Thermoblation is a procedure using heat to remove tissue or a part of the body or reduce the volume of tissue through scarification. To treat sleep apnea, thermoblation may be used to remove or reduce the volume of tissue at various treatment sites of anatomical obstruction, which are known to cause the symptoms of sleep apnea, such as enlarged portions of the inferior nasal turbinates, the soft pallet, the base of the tongue, the lingual tonsil, and the epiglottis. The variable rigidity of the endoscope overtube beneficially allows the physician to insert the endoscope and overtube transnasally and maneuver the endoscope tube through the patient's nasal passageway to various treatment sites, while flexible to maintain the patient's comfort, and then beneficially allows the physician to increase the rigidity of the endoscope overtube once the working end of the endoscope is located at one of the various treatment sites, which allows sufficient force to be transmitted from the proximal end of the ablation catheter to the distal ablation tip for penetration of the affected tissue at the treatment sites. Even further, the variable stiffness of the endoscope overtube allows the physician to access even the deepest layers of tissue at the various treatment sites and reduce that tissue's volume, while keeping the patients mucosa intact, by employing a minimally sized puncture wound into which the ablation catheter is inserted into the patient tissue. Additionally, using the single tool to treat multiple treatment sites allows for faster procedures to be performed transnasally in an office setting as opposed to an operating room environment.

FIG. 8 is a flow chart of a method 800 according to embodiments disclosed herein. The method 800 is a microwave-based tissue ablation/volume reduction method, which may be used in ear, nose and throat procedures, such as procedures for treating sleep apnea. Various devices, such as the endoscope tube 400, may be used to perform the method 600. The method 800 begins at operation 810 by inserting an endoscope through the nasal passage of a patient to a first treatment site. At operation 820, microwaves are delivered to the first treatment site to ablate, or otherwise reduce a volume of, a first tissue at the first treatment site. In one example, the endoscope tube includes an endoscope channel and a working channel. The working channel has a catheter inserted such that its proximal end to the surgeon is connected to a microwave energy source and its distal end is a needle tip or antennae that can deliver microwave energy to the various treatment sites within the patient.

In one embodiment, a variable stiffness endoscope tube, such as the variable stiffness endoscope tube 400 is used to perform the method 800. Accordingly, the method 800 further includes actuating an actuation layer of the variable stiffness endoscope tube to increase the rigidity of the variable stiffness endoscope tube. Additionally, the method 800 can be repeated to treat multiple treatment sites by deactivating the actuation layer of the variable stiffness endoscope tube to decrease the rigidity of the variable stiffness endoscope tube, directing the variable stiffness endoscope tube to a second treatment site, actuating the actuation layer of the variable stiffness endoscope tube to increase the rigidity of the variable stiffness endoscope tube, and delivering microwaves to the second treatment site to ablate a second submucosal tissue at the second treatment site.

As discussed above, in sleep apnea, thermoblation may be used to remove tissue at various treatment sites internal of a patient, the condition of which are known to cause sleep apnea, such as locations on or of the nose, the pallet, the tongue and the epiglottis. The microwave-based ablation method 800 is useful for ablating, or otherwise reducing the volume of, tissue in the submucosal space at each of these sites without damaging the mucosa. Beneficially, microwaves are more tissue agnostic than other radiofrequencies and therefore provide improved ablation of fatty tissues, such as those tissues on or of the nose, the pallet, the tongue and the epiglottis which commonly cause sleep apnea. Additionally, microwaves provide a more reliable and controllable ablation zone from the tip of the catheter. Even further, microwaves provide a more uniform signal, less charring of the tissue at the various treatment sites, less post-procedure pain since there is less contact with the nerves, and less heat loss to the vessels, which are typically a heat sink for conventional ablation methods.

In addition to the ablation procedures, embodiments of variable stiffness endoscope tubes described herein are useful to deliver and deploy balloons for various medical procedures, such as balloon sinuplasty procedures. For example, a balloon sinuplasty method generally includes inserting an endoscope tube through the nasal passage of a patient to a first treatment site, such as a first sinus or turbinate. Once at the first treatment site, an actuation layer of the variable stiffness endoscope tube is actuated to increase the rigidity of the variable stiffness endoscope tube. Then, a balloon is deployed from the variable stiffness endoscope tube to open or dilate the sinus or turbinate site. The balloon may then be retracted and the actuation of the actuation layer may be stopped such that the endoscope tube becomes flexible and can be directed to the next treatment site, such as a second sinus or turbinate site. These operations may be repeated any suitable number of times to complete the balloon dilation procedure. Similarly, the disclosed variable stiffness endoscope tubes can be used to perform balloon dilation at any desired site throughout the body.

Benefits of the present disclosure include, but are not limited to, the ability to make a material or device, such as an endoscope, rigid on demand, and the ability to perform multi-site medical procedures, such as tissue volume reduction, using a single tool in a physician's office. For example, the present disclosure provides a single tool, useful for both access and delivery, which can be inserted transnasally to treat sleep apnea by performing thermoblation at multiple sites, such as the patient's nose, pallet, tongue and epiglottis.

While the foregoing is directed to embodiments of the present disclosure, other and further embodiments of the disclosure may be devised without departing from the basic scope thereof, and the scope thereof is determined by the claims that follow.

What is claimed is:

1. A variable stiffness endoscope assembly, comprising:
a body defining one or more channels; and
an actuation layer disposed around at least one of the one or more channels, the actuation layer comprising a plurality of variable stiffness robotic material cells, each variable stiffness robotic material cell comprising:
a first compression sheet and a second compression sheet; and
a plurality of thin sheets of material arranged in a stack between the first compression sheet and the second compression sheet, each pair of adjacent thin sheets of the plurality of thin sheets having friction therebetween.

2. The variable stiffness endoscope assembly of claim 1, wherein the body is an endoscope overtube.

3. The variable stiffness endoscope assembly of claim 1, wherein the body is an endoscope device.

4. The variable stiffness endoscope assembly of claim 1, wherein the plurality of thin sheets have one or more actuator channels therethrough.

5. The variable stiffness endoscope assembly of claim 4, further comprising:
at least one actuator disposed in each of the one or more actuator channels, wherein the at least one actuator comprises an electropermanent magnet.

6. The variable stiffness endoscope assembly of claim 1, wherein the one or more channels comprises:
an endoscope channel; and
a working channel.

7. The variable stiffness endoscope assembly of claim 6, wherein the working channel further comprises one or more tools selected from the group consisting of a dilation balloon assembly, a needle, forceps, a catheter, and a radiofrequency delivery tip.

8. A variable stiffness endoscope assembly, comprising:
a body defining one or more channels;
at least one of the one or more channels comprising:
a lumen formed by an inner tube manufactured of a flexible material;
a plurality of actuator rings distributed along a length of and attached to an outer surface of the inner tube, each of the plurality of actuator rings having one or more ring channels therethrough; and
one or more tensioned lines running along the length of the inner tube and extending through the one or more ring channels of the plurality of actuator rings, each actuator ring comprising at least one actuator configured to hold and release at least one of the one or more tensioned lines.

9. The variable stiffness endoscope assembly of claim 8, wherein the body is an endoscope overtube.

10. The variable stiffness endoscope assembly of claim 8, wherein the body is an endoscope device.

11. The variable stiffness endoscope assembly of claim 8, further comprising: an outer polymer shell surrounding the lumen, the plurality of actuator rings, and the one or more tensioned lines.

12. The variable stiffness endoscope assembly of claim 8, further comprising: a collet mechanism disposed within each of the one or more ring channels.

13. The variable stiffness endoscope assembly of claim 8, further comprising: a capstan mechanism disposed within each of the one or more ring channels.

14. The variable stiffness endoscope assembly of claim 8, wherein the plurality of actuator rings are manufactured of a shape-memory alloy.

15. A method, comprising:
inserting a variable stiffness endoscope tube through the nasal passage of a patient to a first treatment site, the variable stiffness endoscope tube having an actuation layer comprising a plurality of variable stiffness robotic material cells, to position a distal end thereof proximate the first treatment site;
actuating the actuation layer of the variable stiffness endoscope tube to increase the rigidity of the variable stiffness endoscope tube proximate the first treatment site; and
performing a medical procedure at the first treatment site using the rigid variable stiffness endoscope tube.

16. The method of claim 15, wherein the medical procedure is a thermablation procedure.

17. The method of claim 16, wherein the first treatment site is selected from the group consisting of the nose, pallet, tongue and epiglottis.

18. The method of claim 15, wherein the medical procedure is a balloon sinuplasty.

19. The method of claim 18, wherein the first treatment site is a sinus.

20. The method of claim 15, further comprising:
deactivating the actuation layer of the variable stiffness endoscope tube to decrease the rigidity of the variable stiffness endoscope tube;
directing the variable stiffness endoscope tube a second treatment site;
actuating the actuation layer of the variable stiffness endoscope tube to increase the rigidity of the variable stiffness endoscope tube; and
performing the medical procedure at the second treatment site using the variable stiffness rigid endoscope tube.
